(19) United States
(12) Patent Application Publication
(10) Pub. No.: US 2014/0142591 A1
(43) Pub. Date: May 22, 2014

(54) METHOD, APPARATUS AND A SYSTEM FOR ROBOTIC ASSISTED SURGERY

(71) Applicant: Auris Surgical Robotics, Inc., Redwood City, CA (US)

(72) Inventors: Jeffery B. Alvarez, Redwood City, CA (US); Jian Zhang, Sunnyvale, CA (US); Alisha Seam, Atlanta, GA (US)

(73) Assignee: Auris Surgical Robotics, Inc., Redwood City, CA (US)

(21) Appl. No.: 13/868,769

(22) Filed: Apr. 23, 2013

Related U.S. Application Data
(60) Provisional application No. 61/637,426, filed on Apr. 24, 2012.

(51) Int. Cl. A61B 19/00; A61F 9/007

(52) U.S. Cl. CPC A61B 19/2203; A61F 9/007
USPC 606/130

(57) ABSTRACT

A solution to facilitate robotic assisted surgery is discussed that allows for improved vision and manipulation.

### CROSS-REFERENCE TO RELATED APPLICATIONS

This application claims priority from the U.S. Provisional Application No. 61/637,426 (Attorney Docket No. 41663-704.101), filed Apr. 24, 2012, the entire content of which is incorporated herein by reference.

### BACKGROUND OF THE INVENTION

1. Field of the Invention

The field of the present application pertains to medical devices. More particularly, the field of the invention pertains to an apparatus, system, and method for robotic assisted surgery.

2. Description of the Background Art

Present microsurgical procedures that are currently extremely technique dependent. For example, several existing solutions for eye surgery involve various techniques with lasers and phacoemulsification.

Modern extracapsular cataract surgery is usually performed using a microsurgical technique called phacoemulsfication, whereby the cataract is emulsified with an ultrasonic hand piece and then suctioned out of the eye. Before phacoemulsification can be performed, one or more incisions are made in the eye to allow the introduction of surgical instruments. The surgeon then removes the anterior face of the capsule that contains the lens inside the eye. A phacoemulsification probe is an ultrasonic hand piece with a titanium or steel needle. The tip of the needle vibrates at ultrasonic frequency to sculpt and emulsify the cataract while a pump aspirates particles through the tip. In some techniques, a second fine steel instrument called a chopper is used from a side port to help with chopping the nucleus into smaller pieces. The cataract is usually broken into numerous pieces and each piece is emulsified and aspirated out with suction. The nucleus emulsification makes it easier to aspirate the particles. After removing all hard central lens nucleus with phacoemulsification, the softer outer lens cortex is removed with suction only. As with other cataract extraction procedures, an intraocular lens implant (IOL), is placed into the remaining lens capsule.

One possible improvement to phacoemulsification is a cataract surgery performed with lasers. Femtosecond Laser cataract surgery is rapidly emerging as a potential technology that may allow for improved precision of incision formation and emulsification of the cataract.

Although phacoemulsification and laser-based cataract surgery work well for many patients, these technologies have several shortcomings. For example, phacoemulsification ultrasound probes must propagate ultrasound energy along the length of the probe, from a proximal transducer to a distal tip. This propagation may lead to transmission of ultrasound energy along the probe to tissues in and around the eye that do not benefit from the transmission. Current lens emulsifying probes generate cavitation energy that is initiated within the area of lens nucleus and radiates outwards towards the lens capsule. This places the lens capsule at risk for damage by this energy. Ultrasound probes also tend to generate more heat than would be desirable for a procedure in the eye. Finally, it may be quite difficult to steer an ultrasound probe around corners or bends, due to the mechanical requirements of propagating the ultrasound wave along the entire instrument. In other words, the probe may have to be rigid or at least more rigid than would be desirable.

Probe based lasers have similar drawbacks. They may generate unwanted heat in the eye and are often difficult to control, thus risking damage to important nearby tissues. They also are easily damaged when attempting to navigate tight corners, as fibers in a laser probe may easily break.

Femtosecond laser systems have been devised to assist in the removal of cataracts. These devices are used to create the entry sites through the cornea and sclera into the eye, as well as to remove the anterior face of the capsule. In addition, the femtosecond laser energy can be focused within the lens nucleus itself, and used to “pre-chop” the lens nucleus into a number of pieces that can then be more easily removed with the phacoemulsification probe. However, these lasers can only fragment the center zone of the lens that is visible within the pupil (the iris blocks the peripheral lens from laser energy), so that fracture and removal of the peripheral lens by another method is still necessary. They are costly to own and operate and have the additional drawback of extending operative time.

Therefore, it would be beneficial to have a new method, apparatus, and system for performing surgery for various applications including eye, endoluminal, micro-surgery, and/or other emulsification applications.

### SUMMARY OF THE INVENTION

Embodiments described herein are directed to a new method, apparatus, and system for performing surgery for various applications including eye, endoluminal, micro-surgery, and/or other emulsification applications.

In several embodiments, the claimed subject matter details the following:

1. Robotic Control and Manipulation

2. Registration of Instrument Position in the Operative Field

3. Enhanced Visualization of Tissue Topology

4. Measurement of Applied Forces During Tissue Manipulation

These and other aspects and embodiments will be described in greater detail below, in reference to the attached drawing figures.

### BRIEF DESCRIPTION OF THE DRAWINGS

FIG. 1 is a perspective view of a robotic assisted surgical system, according to one embodiment of the present invention;

FIG. 2 is a perspective view of an apparatus for a GOCT (Global Optical Coherence Tomography) system, according to one embodiment of the present invention;

FIG. 3 is a perspective view of an apparatus for a Tool OCT surface tagging; according to one embodiment of the present invention;

FIG. 4 is a perspective view of the previous embodiments as applied to tissue topology, according to another embodiment of the present invention.

### DETAILED DESCRIPTION OF THE INVENTION

Although certain preferred embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

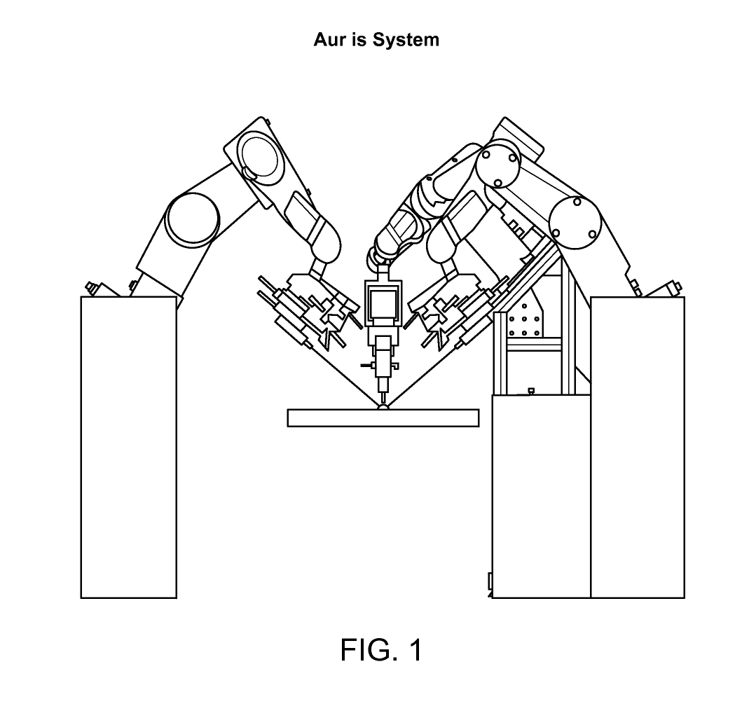

FIG. 1 is a perspective view of a robotic assisted surgical system, according to one embodiment of the present invention.

The robotic platform combines a specially designed manipulation arm with cooperative force control to provide extremely precise, tremor-free motion while preserving much of the transparency and naturalness of conventional instrument manipulation. Force sensors on the robot arm measure surgeon-to-tool interaction forces, and the robot responds by making very precise, tremor-free motions to comply with the surgeon's commands. In one embodiment, the robot arm has 6 degrees of freedom (DOF): three DOFs comprise the Cartesian x, y, z axes, utilized dominantly during system setup where the robot arm and instrument must be positioned in close proximity to the sclera of the eye. The remaining three DOFS, pitch, yaw and roll of the end effector, are used dominantly during instrument manipulation through a pivot point. In the described embodiment, the end effector is an instrument manipulator mechanism that has additional DOF control in the form of both mechanical insertion/retraction and roll, and pneumatic insertion/retraction. Instrument manipulator DOF allows for some instrument motion to be localized to the distal end of the system. Such a configuration reduces the amount of motion required of the robotic arm.

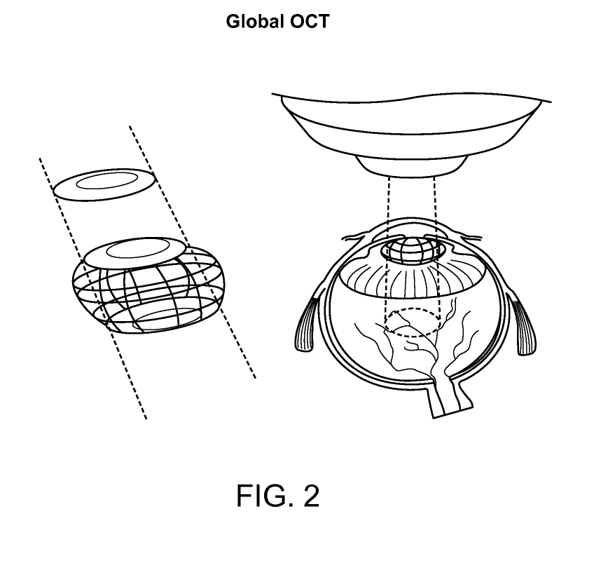

FIG. 2 is a perspective view of an apparatus for a GOCT (Global Optical Coherence Tomography) system, according to one embodiment of the present invention.

The robot “vision arm” is configured to enable 6DOF control of a vision system; a 3D, stereo-optic video system that provides the typical clinical perspective of the surgeon. Integrated with this video system, through shared optics, is an OCT (optical coherence tomography) system that provides additional information to the surgeon in the form of rapid and precise intraocular surface identification. Additionally, the global OCT will establish the world coordinate system to which all arms are registered. This will allow each arm to know their location and the relative location of the other arms and their instruments. An additional embodiment includes a microscopy system for direct surgical visualization by either the operating surgeon or an observing physician.

Global OCT data can be gathered, reconstructed, and presented in a 3 dimensional format either as an overlay onto the imaged anatomy or as a secondary image. This data can further be used to establish surgical boundaries for robotic maneuvers in confined anatomical spaces. Having generated the ocular surface data, which is registered to the GOCT established world coordinate system, and further having instrument tip position in this same coordinate system, an artificial safety barrier can be generated by the robot to maintain a specified minimum distance between the tool and the surface, thereby preventing potential collisions. The robot would simply prevent the tool tip from entering this established ‘keep out’ zone. Another example, based on the tool position information, is an audible warning that can be sounded to alert the physician as the instrument tip approaches within a certain distance of the retinal tissue. Various challenges exist under this boundary establishment modality, namely; shadowing caused by tools, regions of the anatomy obstructed from view and movement of the anatomical target.

Tool shadows occur when imaging light cannot penetrate surfaces that are opaque to its wavelength. In this scenario, an algorithm will identify these shadowed areas and interpolate topology based on topology of the surfaces near the obscured region. The same technique can be used to overcome areas obscured from the global OCT system's view, such as the equatorial regions of the capsule.

Movement of the anatomical surface can be flagged and compensated for by either identifying a shift in the anatomical landmarks using the OCT system or the stereo vision system.

Real-time determination of tool position using the global OCT system is achieved through the use of reflective markers such as retro reflective prisms (similar to those found on street signs), reflective coatings or selective polishing. In the case of retro-reflective markers, light is directed back toward the GOCT system at the inverse of the incidence vector. This results in a strong signal spike or an autocorrelation signal. If a strong signal spike occurs, the distal end of the tip can be identified by location of the signal. If the autocorrelation occurs, the system can identify the tip of the instrument by locating the start of the autocorrelation. In some scenarios, the autocorrelation can saturate the detector, which results in a strong signal through the entire scan. If this were to occur than the tip could not be located in the current embodiment. However, if the instrument had a series identifying retro-reflective strips, which are spaced with various distances between them (similar to bar codes), the configuration of which is also specific to instrument type, than tip location could be determined through kinematic calculations. As before, the strips are recognized in the GOCT system as autocorrelation signatures but, having knowledge of the currently installed instrument and expected autocorrelation positions, the system can determine the exact position and vector of the tool by comparing it to measured autocorrelations. In scenarios where non-linear instruments are utilized, which have the added complexity of sections that change shape, the system must use both a series of retro-reflective strips and a kinematic model of the tool.

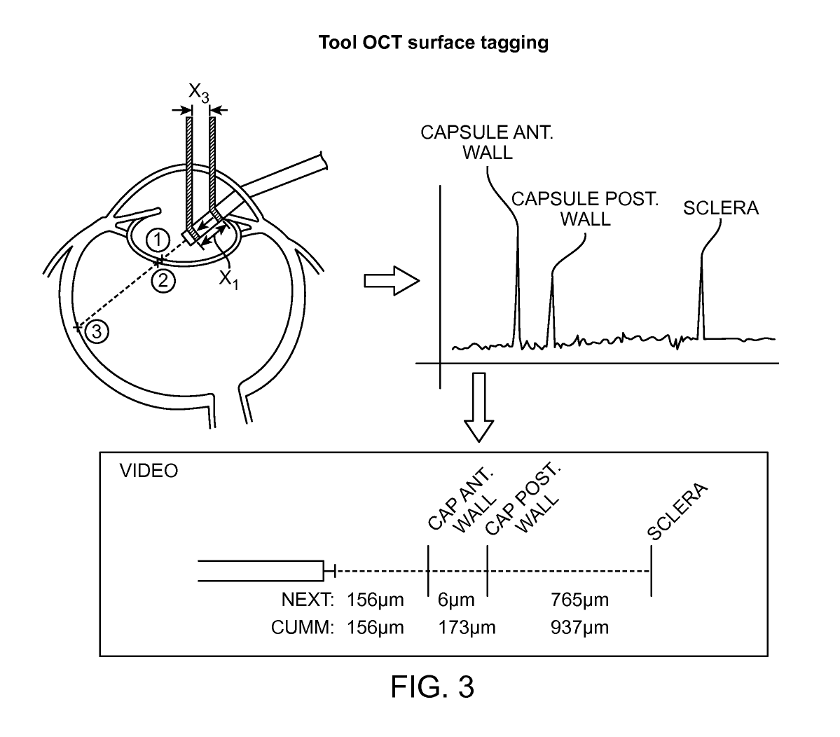

FIG. 3 is a perspective view of an apparatus for a Tool OCT surface tagging; according to one embodiment of the present invention. A tool based OCT system can also be utilized to understand tool position relative to anatomical structures, label imaged structures and monitor for acute changes in anatomy. Such instruments are constructed with embedded fiber optic that images structures directly in front of the instrument tip. Utilizing the instrument position and vector determination described above and correlating this with an anatomical model, signals generated by the TOCT can then be labeled with names and thicknesses. In the example illustrated in FIG. 2, the TOCT is sensing 3 distinct Index of Refraction (IoR) changes; first at the internal capsule wall, then at the external capsule wall, and finally at the internal scleral wall. Each of these surfaces is identified by a peak in the raw TOCT signal. The system can anticipate what surfaces it should see given this instrument position and correlate it with the signals being received. In this case, the system should anticipate both the internal & external capsule walls, and potentially a scleral signal. It can then tag these signals appropriately with labels, thicknesses and distances to tool tip.

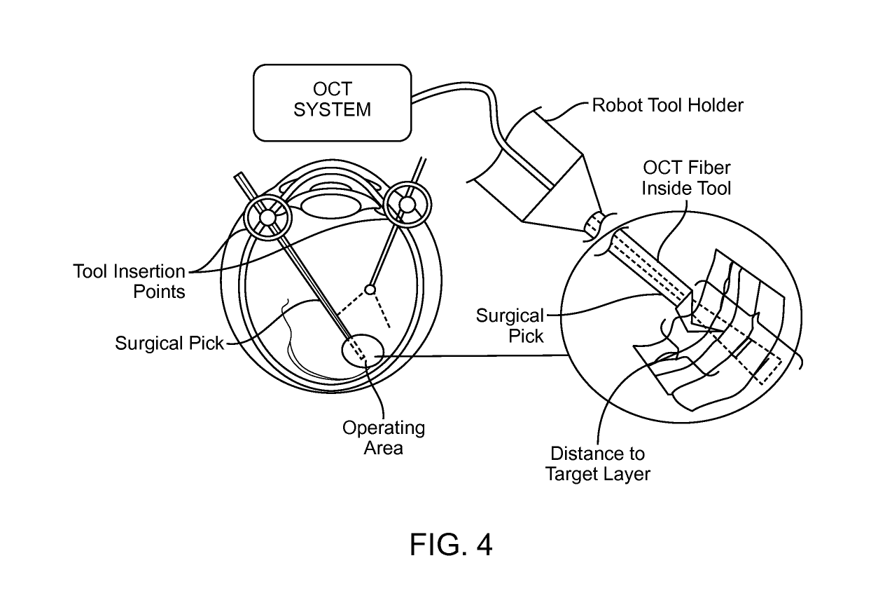

FIG. 4 is a perspective view of the previous embodiments as applied to tissue topology, according to another embodiment of the present invention. Tool based OCT can further be used to enhance the physicians understanding of tissue topology. (.Real-time A, B, and C-Mode images can be used by the surgeon to assess/verify surgical options and procedures. A common issue encountered during retinal surgery is the detection of an ILM edge which the surgeon may grasp for removal of the layer. This can be remedied by utilizing the real-time generated OCT image for ILM edge detection during surgery. Similarly, macular holes and other retinal artifacts may be visualized with this enhanced visualization capability.

Lastly, the tool based OCT can monitor instrument tip proximity to tissue at a rate of 50 Hz. This information can be analyzed for aggressive changes in tissue position relative to robotic manipulation. If an anatomical change occurs that puts the instrument at risk of colliding with either the GOCT establish boundary or the actual anatomy, the robot can withdrawal the instruments to a safe position using the pneumatic system described above.

Elements or components shown with any embodiment herein are exemplary for the specific embodiment and may be used on or in combination with other embodiments disclosed herein. While the invention is susceptible to various modifications and alternative forms, specific examples thereof have been shown in the drawings and are herein described in detail. The invention is not limited, however, to the particular forms or methods disclosed, but to the contrary, covers all modifications, equivalents and alternatives thereof.

What is claimed is:

1. A system for facilitating robotic surgery comprising:
at least two robotic arms to facilitate a surgical application;
one of the robotic arms to enable control of a vision system; wherein
the vision system includes a three dimensional (3D) capability that includes an integrated global OCT (optical coherence tomography) to offer tissue surface identification and the global OCT to establish the world coordinate system to which the robotic arms are registered.

2. The system of claim 1 wherein each robotic arm to be aware of their location and a relative location of the other robotic arm.

3. The system of claim 1 wherein the vision system supports a six degree of freedom.

4. The system of claim 1 to define a boundary for maneuvers of the robotic arms based at least in part on the Global OCT.

5. The system of claim 1 wherein a plurality of Global OCT is presented in a three dimensional format either as an overlay onto the imaged anatomy or as a secondary image.

6. The system of claim 1 further comprising an algorithm for identifying a shadow area from a tool.
